(19) United States
(12) Patent Application Publication
(10) Pub. No.: US 2006/0222660 A1
(43) Pub. Date: Oct. 5, 2006

(54) Use of DNA vaccine in combination with a conventional vaccine to overcome immunogen interference

(75) Inventors: Hsien-Jue Chu, Fort Dodge, IA (US); Yu-Wei Chiang, Fort Dodge, IA (US); Terry Kaleung Ng, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/390,715

(22) Filed: Mar. 28, 2006

Related U.S. Application Data
(60) Provisional application No. 60/667,266, filed on Apr. 1, 2005.

(51) Int. Cl. A61K 48/00; A61K 39/295

(52) U.S. Cl. 424/202.1; 514/44

(57) ABSTRACT

A method, composition and kit for reducing or preventing immunogenic interference in a multi-valent vaccine utilizes a nucleic acid or DNA component along with other non-nucleic acid immunogenic components.

This application claims the benefit of U.S. Ser. No. 60/667,266 filed on Apr. 1, 2005, the entire disclosure of which is incorporated herein by reference.

### FIELD OF THE INVENTION

The present invention relates to methods and compositions for reducing immunogen interference in combination vaccines, and in particular, to methods and compositions which utilize one or more DNA vaccine components together with non-DNA vaccine components to overcome the problems associated with immunogen interaction in multi-valent vaccine preparations.

### BACKGROUND OF THE INVENTION

During the formulation of combination vaccines, i.e. those having more than one immunogenic component, it is not uncommon to encounter immunogen interference in terms of a lowered antibody response in animals vaccinated with the combination vaccine, as compared to those vaccinated with a combination vaccine with fewer components or with monovalent vaccines having only one component. Often times, a lowered antibody response can result in decreased efficacy of one or more of the immunogenic components. This may mean that the immunogen at issue is not being completely effective at soliciting the appropriate immune response. This, in turn, may mean that the animal is not being fully protected against the corresponding disease.

A study to evaluate the immunogenicity of a combination vaccine, containing killed West Nile virus (WNV) as one component, against experimental WNV challenge was evaluated. In this study, the antibody response against WNV as measured by plaque reduction neutralization test (PRNT) (Chang et al., “A Single Intramuscular Injection of Recombinant Plasmid DNA Induces Protective Immunity and Prevents Japanese Encephalitis in Mice”, J. Virol. 74:4244-5422 (2000)) at 14 days post-vaccination was lower than that induced by a monovalent WNV vaccine with equivalent WNV immunogen. Other immunogenic components in the combination vaccine may have caused the reduced antibody response against WNV. Potentially, the WNV immunogens could also have interfered with the immunogenicity or immunogen response of the other components in the combination vaccine.

What is therefore needed in the art is a new method and composition to overcome or alleviate the problem of immunogen interference, particularly in terms of in vivo antibody response. What is also needed is a new approach to address the physical handling of the combination immunogens in a vaccine that will minimize immunogen interference.

### SUMMARY OF THE INVENTION

As part of the invention, there is provided a method of reducing immunogen interference in a vaccine, which comprises administering a DNA vaccine component together with one or more non-DNA vaccine components. Often times, the DNA vaccine component is one which is utilized in place of a more traditional, non-DNA vaccine component against the same disease or affliction.

In addition, there is also provided a method of enhancing the effectiveness of a multi-valent vaccine, which comprises replacing a non-nucleic acid immunogenic component therein with a DNA vaccine component.

The invention is further directed to a method of preventing or reducing a lowered antibody response in an animal upon administration of a multi-valent vaccine preparation, which comprises administering a DNA vaccine component as part of a multi-valent vaccine preparation. The DNA vaccine component may be utilized together with non-DNA or non-nucleic acid vaccine components which otherwise constitute the multi-valent vaccine preparation.

The invention also provides a multi-valent vaccine composition, comprising at least one DNA vaccine component together with at least one non-DNA immunogenic vaccine component.

The invention also sets forth a multi-valent vaccine kit, comprising one or more non-DNA immunogenic vaccine components and at least one DNA vaccine component, wherein said DNA vaccine component is separated from said non-DNA vaccine component. This separation may mean that the vaccine components are in separate containers within the kit, or are separated from each other by means of formulation technology.

Further objects and features of the invention will become apparent from the detailed description and the claims set forth herein below.

### DETAILED DESCRIPTION OF THE INVENTION

In order to reduce or overcome the problem of immunogen interference in multi-valent vaccine preparations, there is provided a method and composition in which a DNA vaccine component or components is included as part of a multi-valent vaccine preparation. The DNA vaccine component may be obtained from whatever source is available to the person skilled in the art. By way of non-limiting example, the DNA vaccine component can include purified DNA, plasmid DNA, or even other nucleic acid components. The invention therefore encompasses all DNA vaccine components derived by technology available in the field, including recombinant technology.

The DNA would encode for the particular immunogenic component of interest against a particular disease or diseases. The immunogenic component could include, for example, proteins which constitute viruses and other disease-causing agents. Expression of the immunogen would then take place in vivo. Of particular interest may be diseases such as viruses which cause disease in veterinary animals, including cows, sheep, pigs, horses, poultry and the like.

The DNA vaccine component as part of the invention would be administered together with other non-DNA vaccine components. These other components would include immunogenic proteins and portions thereof, including peptides and polypeptides, that are derived from what may be considered more traditional or conventional sources, such as via isolation of the particular protein, etc. from an infected animal source.

In a preferred aspect of the invention, the DNA vaccine component is one which is utilized in place of, or as a substitute for, one or more of the more traditional non-DNA vaccine components described in the preceding paragraph. As an example, a viral DNA vaccine component may be used to take the place of an isolated viral immunogen from an infected animal. As a further example, a West Nile DNA vaccine component would be utilized instead of a West Nile conventional isolated immunogen. The West Nile DNA vaccine component could be one which is set forth and described in Chang, US Published Appn. No. 20030022849, which is incorporated herein by reference. It is also within the invention's scope to utilize a rabies DNA vaccine component in place of a conventional immunogen (e.g., peptide or portion thereof) for a rabies vaccine.

It is especially preferred that the DNA vaccine component be substantially equivalent to its corresponding non-DNA vaccine component. This would mean that the DNA vaccine component would elicit substantially the same immunogenic response in vivo. It is also within the scope of the invention that the DNA vaccine component be considered to be more efficacious than its non-DNA counterpart.

The composition of the invention would comprise one or more DNA vaccine components together with one or more non-DNA vaccine components to form a multi-valent vaccine preparation. The DNA vaccine component could be included in the same physical container as the non-DNA vaccine components, or could be included in a separate container. The container or container(s) would then constitute a multi-valent vaccine preparation kit. One or more or all of the vaccine components could be mixed just prior to administration. In an alternative embodiment, where the DNA vaccine component was included together with the other non-DNA vaccine component(s) in the same formulation, then it could be encapsulated. Other formulation technology available to the skilled artisan could be utilized to prevent immunogenic interactions between all the vaccine components.

The vaccine composition of the invention could also comprise one or more adjuvants, or even uptake facilitating components. Preferred adjuvants include SP oil, which is a combination of squalane or squalene, TWEEN 80 and PLURONIC® L 121 surfactants. Also suitable is METASTIM® adjuvant, which is a product of Fort Dodge Animal Health.

The following example is meant to illustrate a preferred aspect of the invention, but should not be construed in any way as limiting the scope thereof.

### Example 1

A new combination vaccine was prepared by mixing one dose of FLUVAC INNOVATOR® vaccine (Fort Dodge Animal Health, Fort Dodge, Iowa), and one dose of WNV DNA vaccine at the time of vaccination. A study was then conducted to evaluate the lack of immunogen blockage on WNV by the other immunogenic components in the conventional vaccine by live WNV challenge.

The combination vaccine, FLUVAC INNOVATOR®, containing encephalomyelitis virus (Eastern, Western and Venezuelan), rhinopneumonitis virus (serotype 1 and 4), influenza virus, and tetanus toxoid was blended with 5% SP Oil in a 1.0 mL dose size. The WNV DNA vaccine containing the purified plasmid as prepared and described in Chang (US 20030022849), was blended to contain 150 μg plasmid DNA and was adjuvanted with 5% SP Oil in a 1.0 mL dose size.

Five horses were vaccinated with FLUVAC INNOVATOR® vaccine while another group of 4 horses received 1 mL of this same conventional vaccine plus 1 mL of the DNA vaccine. For each horse in the combination of the two vaccines, one mL each of vaccine was drawn up in the same syringe and immediately administered to the horse. Vaccines were administered by the intramuscular route in the neck region. A second dose of each vaccine was given three weeks post first vaccination.

Horses in both groups were challenged with a West Nile Virus crow isolate, Lot #V76-2, obtained from Dr. Eilene Ostlund of the CVB-L. The stock challenge material had been stored at −80° C. Serum samples were collected from each animal twice daily for virus isolation starting 0 days post challenge (DPC) until 14 DPC and weekly thereafter until 21 DPC.

Virus isolation results demonstrated (see Table 1) significant protection of horses vaccinated with of a conventional vaccine and DNA vaccine combination against viremia induced by experimental WNV challenge. It is likely that the combination of DNA vaccine and conventional vaccine could overcome immunogen interference when West Nile Virus or other immunogens are involved.

TABLE 1
Viremia Detected by Virus Isolation in Horses Vaccinated with
FLUVAC INNOVATOR ® and West Nile DNA vaccines

| | | Implant | Viremia by virus |
|---|---|---|---|
| Treatment | Horse No. | ID Number | isolation* |
| Inn^{#} + DNA WNV | 4 | 52495F091B | − |
| | 16 | 5246543D1D | − |
| | 14 | 512D09336D | + |
| | 6 | 5317445F44 | − |
| Inn (controls) | 2 | 5317534062 | − |
| | 18 | 53165D6C65 | + |
| | 7 | 52491E2A7A | + |
| | 3 | 5248796406 | + |
| | 12 | 5247663717 | − |

*“+” indicates positive by virus isolation
“−” indicates negative by virus isolation
^{#}“Inn” indicates FLUVAC INNOVATOR ®, a vaccine containing influenza virus, encephalomyelitis virus (Eastern, Western and Venezuelan), rhinopneumonitis virus (serotypes 1 and 4), and tetanus toxoid.
“DNA WNV” indicates vaccine blended with West Nile Virus plasmid DNA

For a clearer understanding of the invention, the foregoing example has been set forth. This example is merely illustrative and is understood not to limit the scope or underlying principles of the invention in any way. Indeed, various modifications of the invention, in addition to those shown and described herein, may become apparent to those skilled in the art from the foregoing description. Such modifications are also intended to fall within the scope of the appended claims, as they are set forth below.

What is claimed is:

1. A method of reducing immunogen interference in a vaccine, which comprises administering a DNA vaccine component together with one or more non-DNA vaccine components.

2. A method of enhancing the effectiveness of a multi-valent vaccine, which comprises replacing a non-nucleic acid immunogenic component therein with a DNA vaccine component.

3. A method of preventing or reducing a lowered antibody response in an animal upon administration of a multi-valent vaccine preparation, which comprises administering a DNA vaccine component as part of said multi-valent vaccine preparation.

4. The method of claim 3, wherein the multi-valent vaccine preparation of said method is formulated for at least one member selected from the group consisting of horses, cows, sheep, pigs, and poultry.

5. The method of claim 4, wherein said member is horses.

6. The method of claim 5, wherein said method comprises administering a West Nile DNA vaccine component together with other non-DNA immunogenic components.

7. The method of claim 6, wherein said non-DNA immunogenic components are selected from the group consisting of influenza virus, encephalomyelitis virus (Eastern, Western and Venezuelan), rhinopneumonitis virus (serotypes 1 and 4), and tetanus toxoid.

8. The method of claim 7, wherein said method further comprises replacing a conventional West Nile immunogenic component with said West Nile DNA vaccine component.

9. A multi-valent vaccine composition, comprising at least one DNA vaccine component together with at least one non-DNA immunogenic vaccine component.

10. The composition of claim 9, wherein said DNA vaccine component is West Nile DNA vaccine component.

11. The composition of claim 10, wherein said composition is suitable for administration to at least one member selected from the group consisting of horses, cows, sheep, pigs and poultry.

12. The composition of claim 11, wherein said composition is suitable for administration to horses.

13. The composition of claim 12, wherein said composition further comprises at least one non-DNA immunogenic component selected from the group consisting of influenza virus, encephalomyelitis virus (Eastern, Western and Venezuelan), rhinopneumonitis virus (serotypes 1 and 4), and tetanus toxoid.

14. The composition of claim 13, wherein said non-DNA immunogenic components comprise influenza virus, encephalomyelitis virus (Eastern, Western and Venezuelan), rhinopneumonitis virus (serotypes 1 and 4), and tetanus toxoid.

15. A multi-valent vaccine kit, comprising one or more non-DNA immunogenic vaccine components and at least one DNA vaccine component, wherein said DNA vaccine component is separated from said non-DNA vaccine component.
